(19) United States
(12) Patent Application Publication
(10) Pub. No.: US 2026/0165381 A1
(43) Pub. Date: Jun. 18, 2026

(54) An Atomizer

(71) Applicant: SHANGHAI QV TECHNOLOGIES CO., LTD., Shanghai (CN)

(72) Inventors: Xiaogang Deng, Shanghai (CN); Guiyou Liu, Shanghai (CN); Xiaofeng Peng, Shanghai (CN)

(21) Appl. No.: 19/125,355

(22) PCT Filed: Oct. 30, 2023
(86) PCT No.: PCT/CN2023/127580

§ 371 Date: Apr. 29, 2025

(30) Foreign Application Priority Data
Nov. 4, 2022 (CN) 202211380428.4

(51) Int. Cl. A24F 40/46; A24F 40/10; A24F 40/42; A24F 40/485

(52) U.S. Cl. CPC A24F 40/46; A24F 40/10; A24F 40/42; A24F 40/485

(57) ABSTRACT

The purpose of the invention is to provide an atomizer which improves the design of air exchanging pathway, and solves the problems of the existing atomizer, including poor air exchange and difficulty in discharging liquid in the application scenario of highly viscous atomizing liquid.

### TECHNICAL FIELD

This invention generally relates to the field of atomization, in particular to an electronic atomizer.

### PRIOR ART

The atomization of this invention refers to atomizing liquid into aerosol which can be inhaled by people through the mouth and nose. Atomization functions widely in medical auxiliary equipment such as atomizing therapeutic apparatus, air quality improvement equipment for home and vehicles, electronic cigarettes and other products. The basic working principle of the atomizer is as follow: When negative pressure is applied to the nozzle, the air outside first enters into the atomizer throng the air inlet, and then flows out of the atomizer from the nozzle. While the air flows through the atomizer, the atomizing liquid flows from the liquid storage to the atomizing element, being heated, gasified and mixes with the air that flows into the atomizer to form an aerosol, then flows out of the atomizer from the suction nozzle.

To ensure the smooth flow of atomizing liquid out of the liquid storage in the process above, it is necessary to balance the air pressure inside and outside the liquid storage, that is, air needs to be constantly supplied into the liquid storage. Therefore, air exchange structure is usually set in the atomizer. A typical air exchange structure, namely, a kind of atomizing element bracket disclosed in patent No. CN216961537U, is shown in FIG. 1, wherein the tortuous groove marked with a is the air exchange structure.

The design disclosed above is only suitable for atomizing liquid of low viscosity, but not for atomizing liquid of high viscosity (more than 10,000 cps at the room temperature of 25° C.). When processing atomizing liquid of low viscosity, it is necessary to increase the resistance to leakage as much as possible in order to prevent atomizing liquid from leaking into the air path. So air exchange structure a is designed as narrow, long and tortuous outside the atomizing element bracket. Because there is no need to worry about the fluidity of condensate and atomizing liquid while processing atomizing liquid of low viscosity, most of the atomizing liquid in the air exchanging pathway would flow back to the liquid storage under the action of pressure even if a small amount of atomizing liquid enters the air exchanging pathway when the pressure and amount of atomizing liquid in the storage decrease. However, in the scenario of highly viscous atomizing liquid, the resistance to air bubble rise is already large, and the narrowness and longness of air path would increase the resistance. Moreover, once the atomizing liquid appears in the inner part again, poor liquidity of the viscous atomizing liquid will block the air path and further increase the resistance to the air bubble, then disable the bubbles to enter the liquid storage via passing through the atomizing liquid. This would cause block to the fluid, which will cause the dry ashing and burnout of atomizing element, harmful substances under high temperature will be released.

### SUMMARY OF THIS INVENTION

In view of the shortcomings of the prior art, the present invention aims to provide an atomizer characterized in that it comprises a suction nozzle, a shell, and a bottom cover; the shell includes a liquid storage, the atomizing element bracket is arranged below the liquid storage, and the atomizing element bracket comprises a liquid inlet; an atomizing element installation bin is arranged below the liquid inlet, and the atomizing element assembly is installed there; at least one air exchanging pathway is arranged between the inner wall of the atomizing element installation bin and the atomizing element assembly; the upper end of the air exchanging pathway is connected with the liquid storage, and its lower end is connected with the air inlet of the atomizer.

Preferably, a groove is arranged on the inner wall of the atomizing element installation bin; the groove and the outer wall of the atomizing element assembly form the air exchanging pathway in the atomizer.

Preferably, a groove is arranged on the outer wall of the atomizing element assembly; the groove and the inner wall of the atomizing element installation bin form the air exchanging pathway in the atomizer.

This invention also provides an atomizer characterized in that it comprises a suction nozzle, a shell, and a bottom cover; the shell includes a liquid storage, the atomizing element bracket is arranged below the liquid storage, and the atomizing element bracket comprises a liquid inlet; an atomizing element installation bin is arranged below the liquid inlet, and the atomizing element assembly is installed in the atomizing element installation bin; the atomizing element assembly comprises an atomizing element and an atomizing element seal; at least one air exchanging pathway is arranged between the atomizing element seal and the atomizing element; the upper end of the air exchanging pathway is connected with the liquid storage, and its lower end is connected with the air inlet of the atomizer.

Preferably, a groove is arranged on the wall of the atomizing element seal facing the atomizing element, thus the air exchanging pathway is formed between the groove and the outer wall of the atomizing element in the atomizer.

Preferably, the air exchanging pathway ranges from 0.015˜0.04 square millimeter by caliber in the atomizer.

Preferably, the part of the air exchanging pathway near the liquid storage is perpendicular to the down-flowing direction of the atomized liquid in the atomizer.

Preferably, the atomizing element assembly is a cuboid in the atomizer.

The atomizer of the invention improves the design of air exchanging pathway, and solves the problems of the existing atomizer, including poor air exchange and difficulty in discharging liquid in the application scenario of highly viscous atomizing liquid.

### BRIEF DESCRIPTION OF THE FIGURES

FIG. 1 is the schematic diagram on the air exchange structure of the atomizer by the prior art;

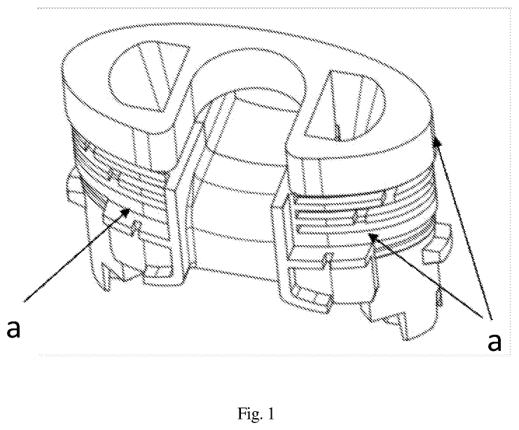

FIG. 2 is the stereogram of an atomizer invented this time;

FIG. 3 is the front view of the atomizer in FIG. 2;

FIG. 4 is a sectional view of the first embodiment along Line C-C in FIG. 3;

FIG. 5 is an exploded view of the atomizing element bracket and atomizing element assembly in FIG. 4;

FIG. 6 is the left view of the atomizer in FIG. 2;

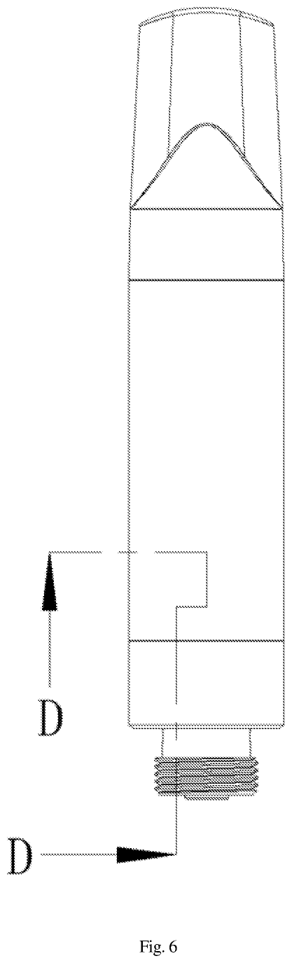

FIG. 7 is an exploded view of the second embodiment along Line D-D in FIG. 6;

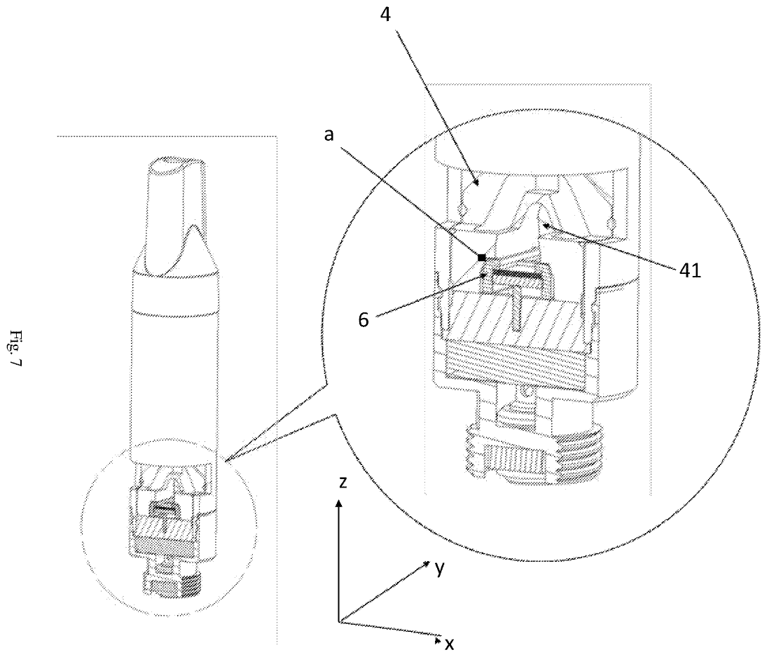

FIG. 8 is an exploded view of the atomizing element bracket and atomizing element assembly in FIG. 7;

FIG. 9 is an exploded view of the atomizing element bracket and atomizing element assembly after the third in embodiment split along Line C-C in FIG. 3;

FIG. 10 is the stereogram of the atomizing element bracket and atomizing element assembly in the fourth embodiment;

FIG. 11 is a sectional view of the atomizing element bracket and atomizing element assembly in the fifth embodiment;

FIG. 12 is a sectional view of the atomizing element assembly in the sixth embodiment.

### EMBODIMENTS

The execution of this invention is explained by specific embodiments below, and a person skilled in the art can easily understand other advantages and effects of this invention from the summary disclosed herein. The invention can also be implemented or applied in other different ways, and the summary herein can also be modified or changed in various ways based on different views and applications without deviating from the spirit of this invention.

Please refer to the attached figures then. To be sure, the figures provided in this embodiment are only explaining the basic conception of this invention, and thus do not show the actual number, size and shape of the component put into practice but only showing the component associated with the invention; the type, number and proportion of the components actually put into practice can be a random change, and the component layout pattern may also be more complex. Unless otherwise specified, the words “up”, “down”, “left”, and “right” used herein are all from the perspective of the observer in the attached figure.

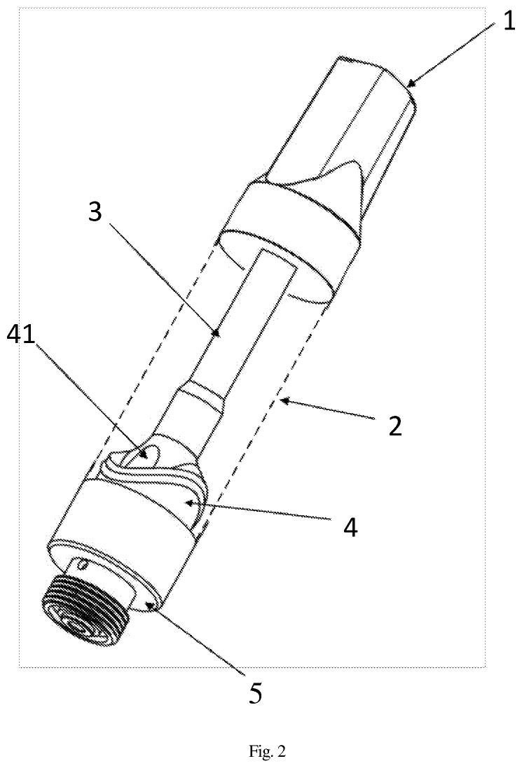

Firstly, please refer to FIG. 2, the structure diagram of the atomizer, which comprises suction nozzle 1, shell 2, outlet passage 3, atomizing element bracket 4, and bottom cover 5. To clearly display the internal structure of the atomizer, shell 2 is represented by a dotted line. The lower end of outlet passage 3 is connected with the air outlet at the top of the atomizing element bracket 4. The outer wall of outlet passage 3, the shell 2, and the upper surface of atomizing element bracket 4 together form a liquid storage for storing the atomized liquid. And the atomizing element bracket 4 comprises liquid inlet 41.

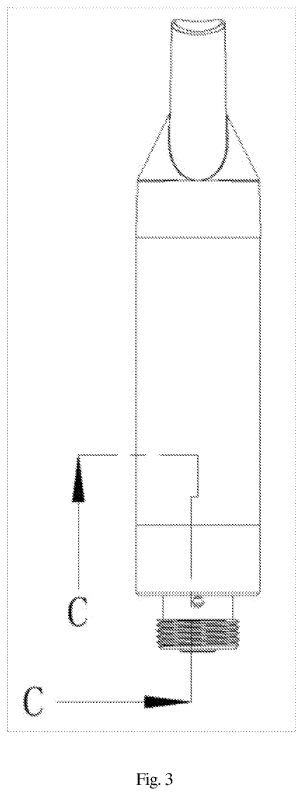

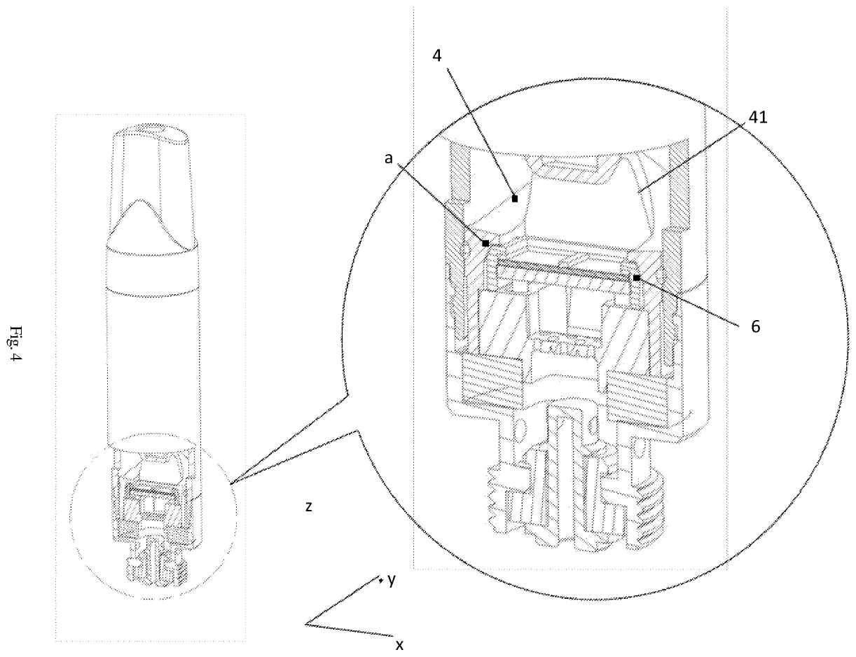

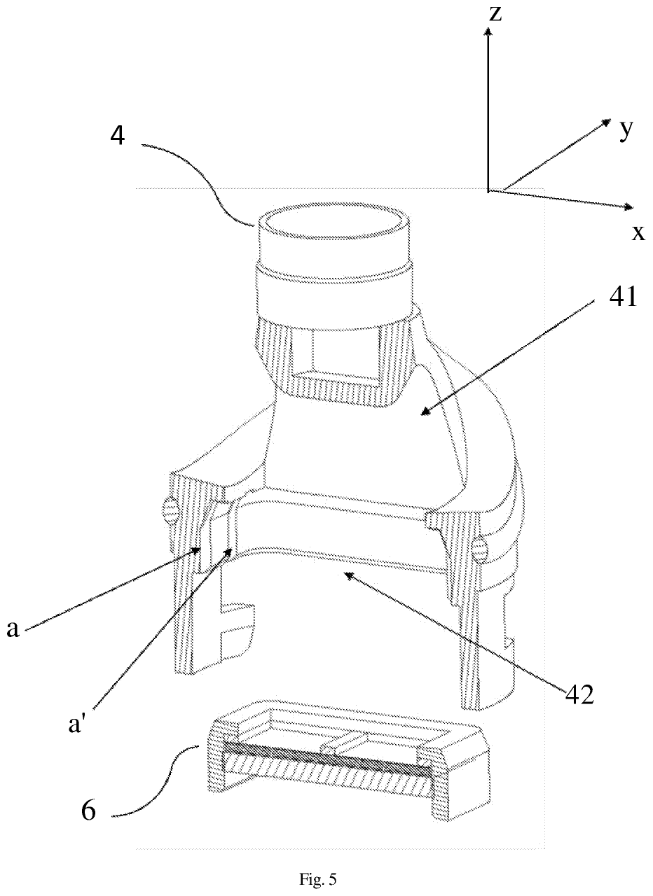

Then please refer to FIG. 3-5. FIG. 4 is the sectional view along Line C-C in FIG. 3, and FIG. 5 is an exploded view of atomizing element bracket 4 and atomizing element assembly 6 in FIG. 4. As shown in the figure, atomizing element installation bin 42 is arranged below atomizing element bracket 4 to install atomizing element assembly 6, which comprises the atomizing element for heating.

In this embodiment, the air exchange structure is also represented by mark a, and arranged between the inner wall of atomizing element bracket 4 and atomizing element assembly 6. The upper end of air exchanging pathway a is connected to liquid inlet 41, thus to the liquid storage, and its lower end is connected to the air inlet of the atomizer. Air exchanging pathway a is arranged between the inner wall of atomizing element installation bin 42 and atomizing element assembly 6. Compared with the prior design in which the air exchanging pathway is arranged outside the atomizing element bracket, the distance between air exchanging pathway a and the heating component is greatly reduced, thus the atomized liquid at the outlet of air exchanging pathway a would be less viscous in the preheating and heating process of the heating component, so that the bubbles in the groove are easy to escape upward.

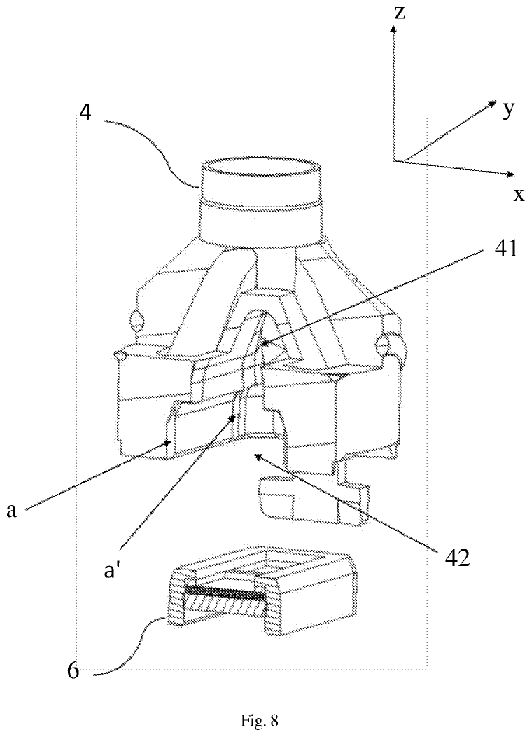

The first embodiment of air exchanging pathway a is shown in FIG. 5. In this embodiment, the atomizing element installation bin is a cuboid, and a groove is set on the short side of the inner wall of atomizing installation bin 42, while the outer surface of atomizing element assembly 6 is flat; in the second embodiment shown in FIG. 8, a groove is provided on the long side of the inner wall of atomizing element installation bin 42. In this way, air exchanging pathway a will be formed between atomizing element assembly 6 and the inner wall of atomizing element installation bin 42. It is understandable that multiple air exchanging pathways can be set up on the inner wall of the installation bin 42, extra air exchanging pathway(s) is(are) represented by mark a′ in FIG. 5 and FIG. 8.

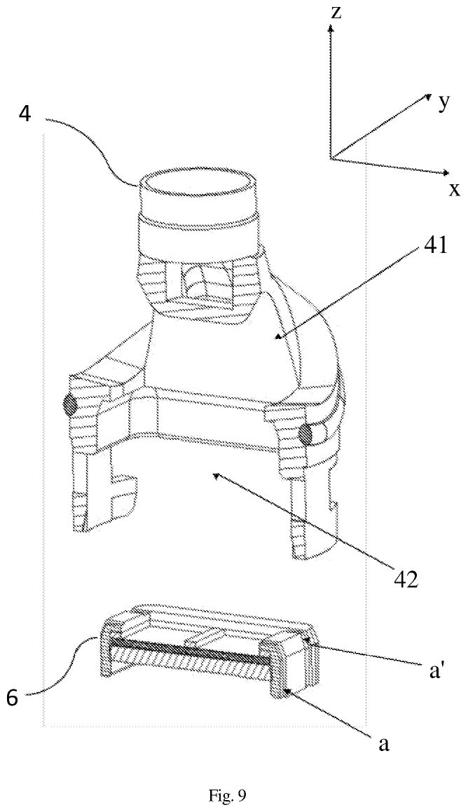

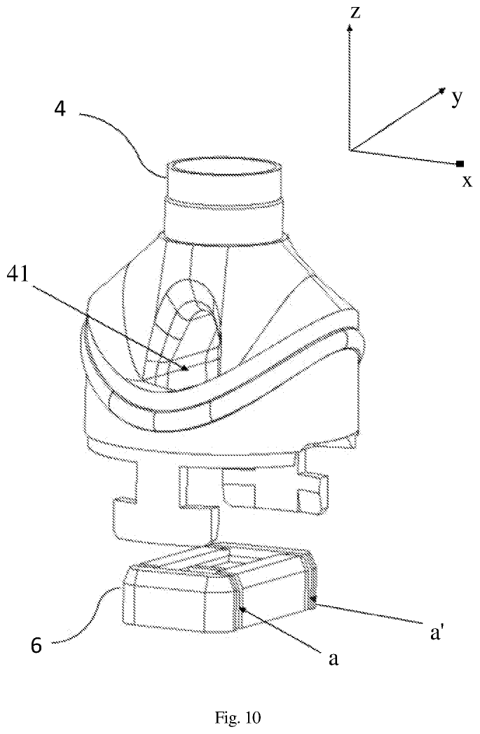

The third and fourth embodiments of air exchanging pathway a are shown in FIG. 9 and FIG. 10, that is, a groove is set on the outer surface of atomizing element assembly 6, and the inner wall of atomizing element installation bin 42 is flat; in the third embodiment, the groove is set on the short side of the outer wall of atomizing element assembly 6; in the fourth embodiment, the groove is set on the long side of the outer wall of atomizing element assembly 6. Then, air exchanging pathway a (and a′) would be formed between atomizing element assembly 6 and the inner wall of atomizing element installation bin 42.

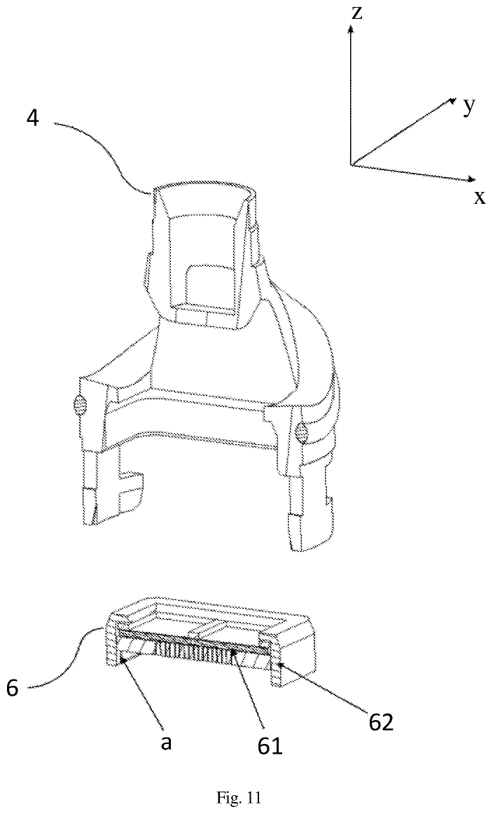

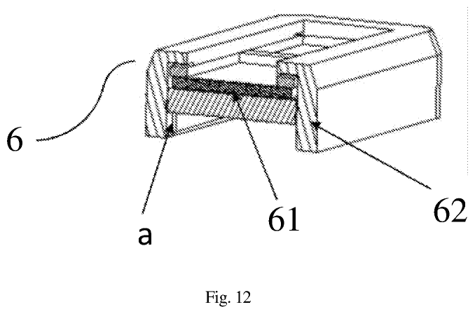

The fifth and sixth embodiments of air exchanging pathway a are shown in FIG. 11 and FIG. 12, wherein, atomizing element assembly 6 comprises atomizing element 61 and seal 62. Groove a is set on the side of seal 62 facing atomizing element 61. In the fifth embodiment, a groove is arranged on the side of seal 62 facing the short side of atomizing element 61; in the sixth embodiment, a groove is arranged on the side of seal 62 facing the long side of atomizing element 61.

As a preferred embodiment, the shape of air exchanging pathway a can also be determined by the intersection line between the vertical plane (x-z in FIG. 4 and FIG. 5) and the inner wall of atomizing element installation bin 42, as shown in FIG. 5 and FIG. 8. In this way, air exchanging pathway a has the shortest length and vertical directivity, the route of bubble filling is shortened, bubble blockage is less possible, the formation of condensate is reduced, and automatic unblocking is achievable even if there is condensate since it can sink away from the air exchanging pathway under the action of gravity. According to the preferred embodiment, multiple air exchanging pathways can be set to further ensure that even if some air exchanging pathways are blocked, the liquid storage can still be successfully filled with air.

Moreover, atomizing element installation bin 42 is a cuboid in the above embodiments, but this does not constitute a limitation. Atomizing element installation bin 42 can be set into other common shapes such as semi-sphere or oval based on actual needs, but the setting mode of air exchanging pathway a is still applicable.

The air exchanging pathway is connected to the liquid storage. In order to avoid liquid leakage, the air exchanging pathway shall be kept away from the liquid down-flowing direction near connection point on the one hand. For example, the part of the air exchanging pathway that near the liquid storage bin is set perpendicular to the direction of liquid down flowing in this embodiment; the caliber of the air exchanging pathway (expressed by its section area) shall be controlled. Generally speaking, the caliber of the air exchanging pathway can be appropriately expanded for the atomizing liquid with higher viscosity. The section of the air exchanging pathway in the present invention is a rectangle, with the long side ranging from 0.3-0.5 mm, and the width from 0.05-0.08 mm. Therefore, the caliber ranges from 0.015-0.04 square millimeter on the other hand. For air exchanging pathways of other section shapes, the above caliber range can be referred to.

The above embodiments are illustrative only of the principle and effectiveness of the invention and are not intended to limit the invention. Any person who is familiar with the technique may modify or alter the above embodiments without prejudice to the spirit and scope of the invention. For example, in the first to fourth embodiments above, grooves are set only on the outer surface of atomizing element assembly 6 or on the inner wall of atomizing element installation bin 42 to form the air exchanging pathway, it is easy to think that grooves can be set on the outer surface of atomizing element assembly 6 as well as on the inner wall of atomizing element installation bin 42, and the grooves are set face-to face to each other, so that they can form an air exchanging pathway together; furthermore, the improvement to the atomizer of this invention is mainly embodied in the air exchanging pathway, the shape and position of the liquid storage, nozzle and other components in the embodiment shall not constitute the limitation of the invention. Therefore, all equivalent modifications or changes made by those with common sense in the technical field to which the invention belongs without prejudice to the spirit and technical ideas revealed by the invention shall still be covered by the claims of the invention.

1. An atomizer comprising:
a suction nozzle,
a shell including a liquid storage,
a bottom cover,
an atomizing element bracket is arranged below the liquid storage, the atomizing element bracket comprising a liquid inlet,
an atomizing element installation bin is arranged below the liquid inlet,
an atomizing element assembly installed in the atomizing element installation bin, and
at least one air exchanging pathway is arranged between an inner wall of the atomizing element installation bin and the atomizing element assembly; an upper end of the air exchanging pathway is connected with the liquid storage, and a lower end thereof is connected with an air inlet of the atomizer.

2. The atomizer of claim 1 is wherein a groove is arranged on the inner wall of the atomizing element installation bin; the groove and the outer wall of the atomizing element assembly form the air exchanging pathway.

3. The atomizer of claim 1 wherein a groove is arranged on the outer wall of the atomizing element assembly; the groove and the inner wall of the atomizing element installation bin form the air exchanging pathway.

4. An atomizer comprising:
a suction nozzle,
a shell including a liquid storage,
a bottom cover,
an atomizing element bracket arranged below the liquid storage, the atomizing element bracket comprises a liquid inlet;
an atomizing element installation bin is arranged below the liquid inlet,
atomizing element assembly installed in the atomizing element installation bin; the atomizing element assembly comprises an atomizing element and an atomizing element seal, and
at least one air exchanging pathway is arranged between the atomizing element seal and the atomizing element; upper end of the air exchanging pathway is connected with the liquid storage, and a lower end thereof is connected with an air inlet of the atomizer.

5. The atomizer of claim 4 wherein a groove is arranged on the wall of the atomizing element seal facing the atomizing element, thus the air exchanging pathway is formed between the groove and the outer wall of the atomizing element.

6. The atomizer of claim 4 wherein the air exchanging pathway ranges from 0.015-0.04 square millimeter by caliber.

7. The atomizer of claim 4 wherein a part of the air exchanging pathway near the liquid storage is perpendicular to the down-flowing direction of the atomized liquid.

8. The atomizer of claim 4 wherein the atomizing element assembly is a cuboid.

9. The atomizer of claim 8 wherein there are four air exchanging pathways.

10. The atomizer of claim 9 wherein a long side or short side of the atomizing element installation bin is provided with two air exchanging pathways.
